(19) United States
(12) Patent Application Publication
(10) Pub. No.: US 2004/0144381 A1
(43) Pub. Date: Jul. 29, 2004

(54) Warmer

(75) Inventor: Bor-Jye Liang, Shichih City (TW)

Correspondence Address: BRUCE H. TROXELL, SUITE 1404, 5205 LEESBURG PIKE, FALLS CHURCH, VA, 22041, US

(73) Assignee: A.C.E.C. Technology Co., Ltd.

(21) Appl. No.: 10/745,599

(22) Filed: Dec. 29, 2003

(30) Foreign Application Priority Data
Jan. 27, 2003 (TW) 092201464

(51) Int. Cl. A61F 7/00

(52) U.S. Cl. 126/208; 431/268

(57) ABSTRACT

A warmer comprising a shell body made of fire-resistant material with a space and absorber made of none spontaneous combustion or none combustion supporting materials with many small holes or slots, the absorber is in the space of the shell body; flammable organic fuel is filled into the shell body for the absorber to absorb, a other burning source ignites the fuel for red smolder condition to generate continuous heat for warming effect.

### BACKGROUND OF THE INVENTION

1. Field of the Invention

This invention relates generally to a warmer and, more specifically, to a warmer in shell body shape filled with absorber made of none spontaneous combustion or none combustion supporting materials, flammable organic fuel is filled into the body shell for the absorber to absorb, a fire source ignites the fuel for red smolder condition to generate continuous heat for warming effect.

2. Description of the Prior Art

Heretofore, it is known that a warmer, a pocket warmer for example, is to have a bundle of cotton in an empty shell body for fuel to fill in, a wick in the bundle of cotton expands to the heating area with a catalyzer (platinum coil), a heating wire is on top of the catalyzer, the current of a battery heat up the heating wire and trigger the catalyzer to smolder, the heat is transfer to the shell body.

The pocket warmer apply capillarity to have fuel transfer from cotton to catalyzer through wicks, the catalyzer can smolder inside the shell body. The cotton of the pocket warmer only absorbs fuel and not for oxidation of burning. The catalyzer is the origin of smolder, without the catalyzer, the pocket warmer is obsolete.

### SUMMARY OF THE INVENTION

It is therefore a primary object of the invention to provide a warmer comprising a shell body filled with absorber made of none spontaneous combustion or none combustion supporting materials, flammable organic fuel is filled into the shell body for the absorber to absorb, a fire source ignite the flammable organic fuel to make the absorber in red smolder condition to generate continuous heat for warming effect.

It is an objective of this invention to provide a warmer in which a catalyzer is on the absorber to increase the smolder effect. At least one scent additive is in the flammable organic fuel to offer mild scent during smolder.

It is still an objective of this invention to provide a warmer system in which a burning source is on the absorber. The burning source can also be in between the absorber and the catalyzer.

### BRIEF DESCRIPTION OF THE DRAWINGS

The accomplishment of the above-mentioned object of the present invention will become apparent from the following description and its accompanying drawings which disclose illustrative an embodiment of the present invention, and are as follows:

FIG. 1. is a cross-sectional view of the present invention;

FIG. 2 is a cross-sectional view of the second application of the present invention;

FIG. 3 is a cross-sectional view of the third application of the present invention; and

FIG. 4 is a cross-sectional view of the fourth application of the present invention.

### DESCRIPTION OF THE PREFERRED EMBODIMENT

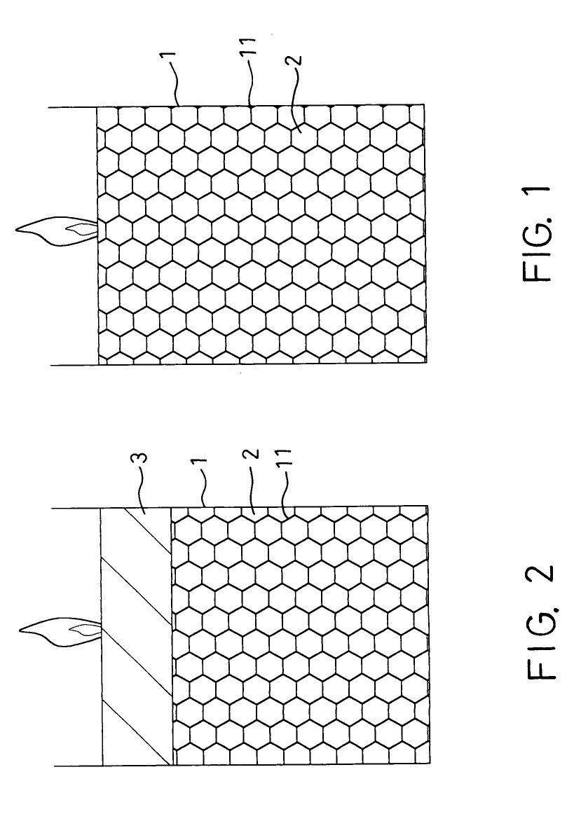

Referring to FIG. 1, the present invention is composed of a shell body 1 and the absorber 2. The functions of each component are described below:

The shell body 1 is made of fire-resistant material, a space 11 is for the absorber 2 to fill in.

The absorber 2 is made of none spontaneous combustion or none combustion supporting materials with many small holes or slots, such as ceramic, calcium carbonate, fiber optic, zeolite, and so on, the shape of the absorber 2 is in flake, in chunk in pellet, or in particle, the purpose is to have the absorber 2 with small holes or slots to absorb flammable organic fuel, and have the absorber 2 participate the oxidation of burning to form the red smolder, after the burning the absorber 2 still remain the same without loss.

Referring to FIG. 1, while in use, flammable organic fuels, such as methanol, alcohol, WD40, oil, gasoline is filled into the shell body 1 and have the absorber 2 soak adequately, then apply a fire source, such as a piezo-electric device, to generate heat to start up the catalyzer on the absorber 2 and form enough heat to generate continuous warming heat.

In order to have scent effect, the flammable organic fuel might contain at least one scent additive, such as perfume, essence, scent grease or insect repellent, and so on, to increase mild scent while the absorber 2 is under smolder.

Referring to FIG. 2 that shows another application of the present invention, the difference from above application is a catalyzer 3 covers the absorber 2, such as filament bundle, honeycomb, particles or irregular shape cover made of platinum and/or rhodium and/or palladium . . . , the purpose is to have the catalyzer 3 increase red smolder effect for better oxidation.

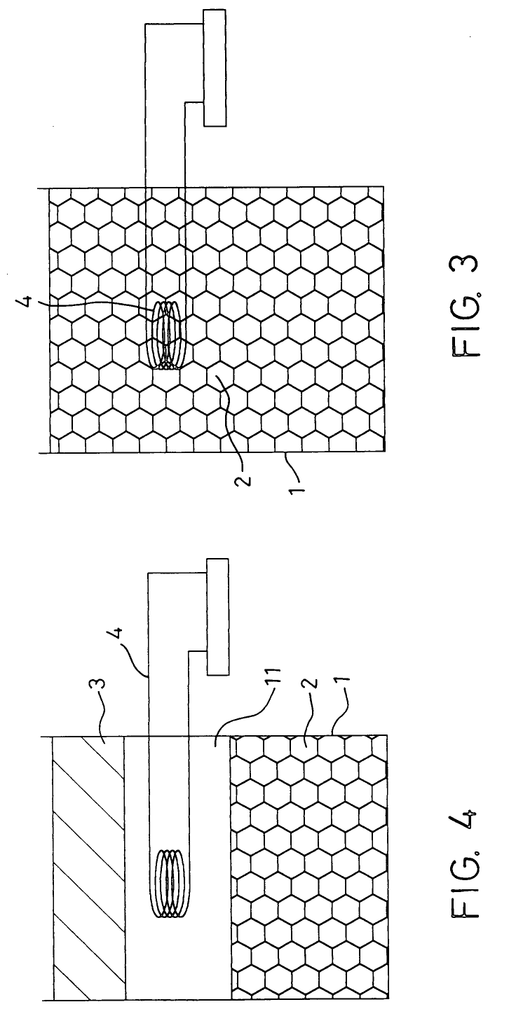

Referring to FIG. 3 that shows a third application of the present invention, the difference from the first application is a burning source 4, such as heating wires, is on the absorber 2, by the ignition of burning source 4, the absorber 2 with flammable organic fuel can achieve red smolder effect.

Referring to FIG. 4, the burning source 4 is installed between the absorber 2 and the catalyzer 3, such arrangement can also achieve red smolder effect as the absorber 2 and the catalyzer 3.

The absorber of the present invention not only absorbs flammable organic fuel but also participates oxidation of burning, when the absorber is in red smolder condition, heat is transmitted continuously for expected warming effect. The scent additives can generate mild scent during burning. The present invention can also have catalyzer and burning source work together, and build pocket type or stationary type warmer for different purposes.

While a preferred embodiment of the invention has been shown and described in detail, it will be readily understood and appreciated that numerous omissions, changes and additions may be made without departing from the spirit and scope of the invention.

What is claimed is:

1. A warmer comprising:
a shell body made of fire-resistant material with a space;
a absorber made of none spontaneous combustion or none combustion supporting materials with many small holes or slots is filled into said space, flammable organic fuel is filled into said shell body for said absorber to absorb, a other burning source ignite said absorber for red smolder condition and generate heat continuously.

2. The warmer according to claim 1, wherein said absorber is made of ceramic, calcium carbonate, fiber optic, zeolite, and other same effect material.

3. The warmer according to claim 1, wherein flammable organic fuel is methanol, alcohol, WD40, oil, gasoline, and other same effect material.

4. The warmer according to claim 1, wherein flammable organic fuel contains at least one scent additive.

5. The warmer according to claim 4, wherein scent additive is perfume, essence, scent grease or insect repellent.

6. The warmer according to claim 1, wherein said absorber is covered by a catalyzer.

7. The warmer according to claim 6, wherein said catalyzer is in filament bundle, honeycomb, particles or irregular shape cover and is made of platinum, and/or rhodium and/or palladium.

8. The warmer according to claim 1, wherein a burning source is on said absorber.

9. The warmer according to claim 1, wherein a burning source is between said absorber and said catalyzer.

10. The warmer according to claim 8, wherein said burning source is a heating wire.

11. The warmer according to claim 9, wherein said burning source is a heating wire.
